(19) United States
(12) Patent Application Publication
(10) Pub. No.: US 2008/0160522 A1
(43) Pub. Date: Jul. 3, 2008

(54) Primer for Detecting Food Poisoning and Method for Rapid Detection of Food Born Pathogene

(75) Inventors: Gang-Gweon Lee, Gyeonggi-do (KR); Yong-Min Park, Gyeonggi-do (KE); Jung-Ran Park, Seoul (KR)

Correspondence Address: LEXYOUME IP GROUP, LLC, 1233 TWENTIETH STREET, N.W., SUITE 701, WASHINGTON, DC, 20036, US

(73) Assignee: Samsung Everland Inc., Seoul (KR)

(21) Appl. No.: 11/817,301

(22) PCT Filed: Mar. 15, 2005
(86) PCT No.: PCT/KR05/00727

§ 371 Date: Aug. 28, 2007

(30) Foreign Application Priority Data
Feb. 28, 2005 (KR) 10-2005-0016633

(51) Int. Cl. C12Q 1/68; C07H 21/04

(52) U.S. Cl. 435/6; 536/24.33

(57) ABSTRACT

The present invention relates to a primer of detecting a food-borne pathogen, and to a method of detection for food poisoning. In particular, the invention relates to a PCR primer which is specific to and is used for rapid and accurate detection of the Salmonella spp., Staphylococcus aureus, E. coli O-157, Listeria monocytogenes, and Vibrio parahemoliticus, respectively, and a detection method and a kit for the Food poisoning by using the PCR primer. Using the detection method according to the present invention, 100 to 10 CFU/ml of food borne pathogen can be detected, and rapid survey of food poisoning research can be performed within five (5).

### TECHNICAL FIELD

The present invention relates to a primer for detecting a food-borne pathogen, and to a method of detection for food poisoning. More specifically, it provides a rapid and accurate method for the detection of pathogen that causes food poisoning, using a PCR primer.

### BACKGROUND ART

Food poisoning is an illness that is accompanied by fever, nausea, vomiting, diarrhea, abdominal pain, and is mostly bacterial food poisoning. The bacterial food poisoning can be classified into infective or toxin-caused depending on the pathogenesis.

Infective food poisoning is caused by ingesting food contaminated with bacteria and multiplication of the bacteria in the gastrointestine. The main bacteria causing infective food poisoning are Salmonella spp, Vibrio paraheamolyticus, Escherichia coli O157:H7, and etc.

The toxin produced by bacteria multiplying in the food causes toxin-caused food poisoning. Thus, even after the bacteria in the food are dead, any remaining toxin can cause food poisoning. Examples of such bacteria are Staphylococcus aureus, and Clostridium botulinum, etc.

The number of bacteria required to cause food poisoning is dependent on the type of bacteria. Usually, at least 10⁶ to 10⁸ CFU/g of bacteria is required to cause food poisoning. However, only 10˜1000 of E. coli O157:H7, or Listeria monocytogenes can cause food poisoning. The types and number of bacteria which cause food poisoning are summarized in Table 1.

TABLE 1

| | | | The amount |
|---|---|---|---|
| | | The number of pathogen required to cause | of toxin in |
| Type | Pathogen | disease | food |
| infective | Salmonella spp. | 10⁵-10⁷ cell/g: onset in humans | |
| | | tens of cell/g: infection to newborns | |
| | Vibrio paraheamolyticus | 10⁴-10⁷ cell/g: onset in humans | |
| | Campylobacter jejuni | 10² cell/g: in human(volunteer) | |
| | | 10⁶ cell/g; in human (laboratory infection) | |
| | Listeria monocytogenes | a few cells | |
| | E. coli O-157: H7 | 10~100 cells | |
| Toxin- | Staphylococcus aureus | 10⁶-10⁷ cell/g in food | 1.0 μg/1man |
| caused | | | in food |
| Other | Clostrium perfringens | 10⁸-10⁹ cell/g: onset in humans | |
| | Bacillus cereus | diarrhea: 10⁷-10⁸ cell/g: onset in | |
| | | humans | |
| | | vomiting: 10⁶-10⁷ cell/g in food | |

Food poisoning is on the increase and tends to occur as mass outbreaks on a large scale as dining patterns change where meals provided by institutions and dining out become more widespread, and in there are environmental changes such as global warming, and increase in average room temperatures. The development of a method to detect pathogens causing food poisoning early on in order to prevent food poisoning is urgently needed.

The detection method currently used for Salmonella spp. comprises the first verification step of culturing the bacteria in selective media following culture in Buffered Peptone Water (BPW), the second verification step of biochemical and serological analysis, and normally takes about 5 to 6 days to identify the pathogen. Furthermore, the PCR method used for rapid detection of the pathogen takes 1 day or longer to detect because the amount of pathogen that can be analyzed must be obtained by culturing the sample.

The development of a rapid method to detect pathogens causing food poisoning that does not require a culture process for the pathogen is urgently needed.

### DISCLOSURE

### Technological Problem

To resolve the problems in the prior art, the objective of the present invention is to provide a primer to selectively amplify a specific gene of the pathogen causing food poisoning in humans.

It is another objective of the present invention to provide two pairs of primers with high detection accuracy and specificity for the pathogen causing food poisoning in humans.

It is another objective of the present invention to detect a pathogen accurately, and to shorten a diagnosis time of 4 to 6 days to 12 hours by using PCR with a primer to selectively amplify a specific gene of the pathogen causing food poisoning in humans.

It is another objective of the present invention to provide a kit for detecting the pathogen accurately in a short time by using PCR with a primer to selectively amplify a specific gene of the pathogen causing food poisoning in humans.

### TECHNOLOGICAL SOLUTION

To achieve the objectives of the present invention, the present invention provides at least one pair of primers for detecting a pathogen selected from the group consisting of pairs of primers in the following 1) to 5):

1) a pair of primers for detecting Salmonella spp. comprising primers in SEQ ID NO: 3 and 4;

2) a pair of primers for detecting Staphylococcus aureus comprising primers in SEQ ID NO: 7 and 8;

3) a pair of primers for detecting E. coli O157:H7 comprising primers in SEQ ID NO:11 or 13 and in SEQ ID NO:12 or 14;

4) a pair of primers for detecting Listeria monocytogenes comprising primers in SEQ ID NO:17 and 18; and

5) a pair of primers for detecting Vibrio parahaemolyticus comprising primers in SEQ ID NO:21 or 23 and in SEQ ID NO:22 or 24.

In addition, the present invention provides a method of detecting a pathogen with PCR using at least one pair of primers for detecting a pathogen selected from the group consisting of pairs of primers in the above 1) to 5) which can amplify the specific genes of five (5) kinds of pathogens.

In the method for detecting a pathogen by said PCR, the present invention can use at least one additional pair of primers selected from the group consisting of pairs of primers in the following 6) to 10):

6) a pair of primers for detecting Salmonella spp. comprising primers in SEQ ID NO: 1 and SEQ ID NO: 2;

7) a pair of primers for detecting Staphylococcus aureus comprising primers in SEQ ID NO: 5 and in SEQ ID NO: 6;

8) a pair of primers for detecting E. coli O157:H7 comprising primers in SEQ ID NO:9 and in SEQ ID NO:10;

9) a pair of primers for detecting Listeria monocytogenes comprising primers in SEQ ID NO:15 and 16; and

10) a pair of primers for detecting Vibrio parahaemolyticus comprising primers in SEQ ID NO:19 and in SEQ ID NO:20.

The additional pair of primers is used in the first round of amplification of nested PCR, and remarkably improves the detection limit for pathogen with higher PCR efficiency. In case of nested PCR using an additional pair of primers, PCR can be done in two steps, or in one step simultaneously using the two kinds of primer pairs used in the above two steps.

In addition, the present invention provides a kit for detecting a pathogen by PCR, comprising a PCR primer for detecting a pathogen, a reaction buffer solution, and Taq DNA polymerase.

### ADVANTAGEOUS EFFECT

As explained above, the primer pairs for detecting the pathogen of this invention amplifies a specific gene of Salmonellas spp., Staphylococcus aureus, E. coli O157, Listeria monocytogenes and Vibrio parahaemoliticus accurately in a short time, and with a detection limit of 100 to 10 CFU/ml. Therefore, using the primer pairs, rapid epidemiological investigation of an occurrence of infection can be performed within 5 hours.

### DESCRIPTION OF DRAWINGS

FIG. 1 is an electrophoretic result of nested PCR of Salmonella enteritidis (KCCM12021).

FIG. 2 is an electrophoretic result of nested PCR of Staphylococcus aureus (KCCM1927).

FIG. 3 is an electrophoretic result of nested PCR of Escherichia coli O157:H7 (ATCC12024).

FIG. 4 is an electrophoretic result of nested PCR of Listeria monocytogenes (ATCC19112).

FIG. 5 is an electrophoretic result of nested PCR of Vibrio parahaemolyticus (KCCM11965).

FIG. 6 is an electrophoretic result showing a reaction specificity of a pair of primers for detecting Salmonella sp. Shown in SEQ ID NO:3 and 4.

FIG. 7 is an electrophoretic result showing a reaction specificity of a pair of primers for detecting Staphylococcus aureus shown in SEQ ID NO:7 and 8.

FIG. 8 is an electrophoretic result showing a reaction specificity of a pair of primers for detecting Listeria monocytogenes shown in SEQ ID NO:15 to 18.

FIG. 9 is an electrophoretic result of detection of Salmonella in various foods that were intentionally contaminated in the lab.

FIG. 10 is a comparison of the result of PCR using a pair of primers shown in SEQ ID NO:1 and 2, with the result of micro-PCR using two pairs of primers shown in SEQ ID NO: 1 to 4.

FIG. 11 is an electrophoretic result of nested PCR of Salmonella enteritidis using two pairs of primers shown in SEQ ID NO:1 to 4.

FIG. 12 is an electrophoretic result of micro-PCR of Salmonella enteritidis using a pair of primers shown in SEQ ID NO:3 to 4.

FIG. 13 is an electrophoretic result of micro-PCR of Staphylococcus aureus using a pair of primers shown in SEQ ID NO:7 to 8.

FIG. 14 is an electrophoretic result of micro-PCR of Escherichia coli O157:H7 using a pair of primers shown in SEQ ID NO:11 and 12.

### BEST MODE

The present invention will be described in more detail below.

The present invention relates to a method of detection of a pathogen that causes food poisoning. The pathogen can be one or more selected from the group consisting of Salmonella spp, Staphylococcus aureus, E. coli O157:H7, Listeria monocytogenes, and Vibrio parahaemolyticus.

The present invention provides a pair of primers for amplifying a specific gene of the pathogen in order to detect the specific gene of the pathogen.

The pair of primers of the present invention can simultaneously detect in different nucleotide sizes the specific genes of five (5) kinds of pathogens. Examples are as follows and sequences are listed in Table 2. The primer pairs of the present invention can be used in widely known PCR methods such as nested PCR or micro-PCR.

TABLE 2

| | | | |
|---|---|---|---|
| | | | SEQ |
| | | | ID |
| Pathogen | Primer | Nucleic acid sequence | NO |

| | | | | |
|---|---|---|---|---|
| Salmonella spp. | 1st | P1 | 5′-GAATCCTCAGTTTTTCAACGTTTC-3′ | 1 |
| | | P2 | 5′-TAGCCGTAACAACCAATACAAATG-3′ | 2 |
| | 2nd | P′1 | 5′-TCGTCATTCCATTACCTACC-3′ | 3 |
| | | P′2 | 5′-ATCGGCTTCAATCAAGATAA-3′ | 4 |
| Staphylococcus | 1st | P3 | 5′-AATTTAACAGCTAAAGAGTTTGGT-3′ | 5 |
| Aureus | | P4 | 5′-TTCATTAAAGAAAAAGTGTACGAG-3′ | 6 |
| | 2nd | P′3 | 5′-AATGCTTTCCGTCATTTTGC-3′ | 7 |
| | | P′4 | 5′-AGCTTTTGCTGATCGTGATG-3′ | 8 |
| E. coli O157:H7 | 1^{st} | P5 | 5′-GACAGCAGTTATACCACTCTGCAA-3′ | 9 |
| | | P6 | 5′-GACGAAATTCTCTCTGTATCTGCC-3′ | 10 |
| | 2^{nd} | P′5 | 5′-GGTGTTCCTTTTGGCTGAAG-3′ | 11 |
| | | P′6 | 5′-TGACGACTGATTTGCATTCC-3′ | 12 |
| | 2^{nd} | P″5 | 5′-TTCTGAGCAATCGGTCACTG-3′ | 13 |
| | | P″6 | 5′-TATATCAGTGCCCGGTGTGA-3′ | 14 |
| Listeria | 1^{st} | P7 | 5′-CTGGCACAAAATTACTTACAACGA-3′ | 15 |
| monocytogenes | | P8 | 5′-AACTACTGGAGCTGCTTGTTTTTC-3′ | 16 |
| | 2^{nd} | P′7 | 5′-TTGGTGCAACTGGAGTGCTT-3′ | 17 |
| | | P′8 | 5′-AGCAGGTGCAGCTTGTTGAG-3′ | 18 |
| Vibrio | 1^{st} | P9 | 5′-CTCATTTGTACTGTTGAACGCCTA-3′ | 19 |
| parahaemolyticus | | P10 | 5′-AATAGAAGGCAACCAGTTGTTGAT-3′ | 20 |
| | 2^{nd} | P′9 | 5′-CTTCTGACGCAATCGTTGAA-3′ | 21 |
| | | P′10 | 5′-GACCTGCGAAAATACGCAAT-3′ | 22 |
| | 2^{nd} | P″9 | 5′-TGATTTGCGGGTGATTTACA-3′ | 23 |
| | | P″10 | 5′-GAGATTCCGCTGGGTTTGTA-3′ | 24 |

A pair of primers for detecting Salmonella spp. consists of a pair of primers shown in SEQ ID NO:3 and 4, and amplify about 200 base pairs of gene product specific to Salmonella spp. In case of nested PCR, a pair of known primers for Salmonella spp. comprising primers in SEQ ID NO:1 and 2 can be reacted simultaneously with the above primer pair, or reacted in a first round of amplification then the product reacted with the above primer pair for detecting Salmonella spp in a second round. The pair of primers comprising SEQ ID NO:1 and 2 amplify about 678 base pairs of the specific gene.

The pair of primers for detecting Staphylococcus aureus shown in SEQ ID NO: 7 and 8 amplify about 136 base pairs of gene product specific to Staphylococcus aureus. For nested PCR, a pair of known primers for Staphylococcus aureus comprising primers in SEQ ID NO:5 and 6 can be reacted simultaneously with the above primer pair, or reacted in a first round of amplification then the product reacted with the above primer pair for detecting Staphylococcus aureus in a second round. The pair of primers comprising SEQ ID NO:5 and 6 amplify about 678 base pairs of the specific gene.

A pair of primers for detecting E. coli O157 consists of a sense primer shown in SEQ ID NO: 11 or 13, and an anti-sense primer shown in SEQ ID NO: 12 or 14. For example, the pair of primers for detecting E. coli O157 includes primers shown in SEQ ID NO:11 and 12 amplifying about 108 bp of a gene specific to E. coli O157, and primers shown in SEQ ID NO: 13 and 14 amplify about 129 bp of the gene specific to E. coli O157.

For nested PCR, a pair of known primers comprising SEQ ID NO:9 and 10 can be reacted simultaneously with the above primer pair, or reacted in a first round of amplification then the product reacted with the above primer pair for detecting E. coli O157 in a second round. The pairs of primers shown in SEQ ID NO:9 and 10 amplify about 208 base pairs of the specific gene.

The pair of primers for detecting Listeria monocytogenes shown in SEQ ID NO: 17 and 18 amplify about 191 base pairs of gene specific to Listeria monocytogenes.

For nested PCR, a pair of known primers for detecting Listeria monocytogenes comprising SEQ ID NO:15 and 16 can be reacted simultaneously with the above primer pair, or reacted in a first round of amplification then the product reacted with the above primer pair for detecting Listeria monocytogenes in a second round. The pairs of primers shown in SEQ ID NO:15 and 16 amplify about 454 base pairs of gene specific to Listeria monocytogenes.

A pair of primers for detecting Vibrio parahaemolyticus consists of a sense primer shown in SEQ ID NO: 21 or 23, and an anti-sense primer shown in SEQ ID NO: 22 or 24. For example, The pair of primers for detecting Vibrio parahaemolyticus includes primers shown in SEQ ID NO:21 and 22 amplifying about 219 bp of a gene specific to Vibrio parahaemolyticus, and primers shown in SEQ ID NO: 23 and 24 amplify about 153 bp of the gene specific to Vibrio parahaemolyticus.

For nested PCR, a pair of known primers for detecting Vibrio parahaemolyticus comprising SEQ ID NO:19 and 20 can be reacted simultaneously with the above primer pair, or reacted in a first round of amplification then the product reacted with the above primer pair for detecting Vibrio parahaemolyticus in a second round. The pairs of primers shown in SEQ ID NO:19 and 20 amplify about 678 base pairs of the gene product specific to Vibrio parahaemolyticus.

A method of the present invention can not only detect each pathogen using a pair of PCR primers for one (1) kind of pathogen, but can also detect two (2) or more kinds of the above pathogens through multiplex PCR using two (2) or more pairs of PCR primers to rapidly and accurately detect the five (5) kinds of the pathogen at once.

The PCR reaction conditions of the method of detection of the present invention are general PCR reaction conditions of the various types of PCR (e.g. nested PCR, multiplex PCR, micro-PCR, single PCR) or partial variations thereof, and within the range of variations that can easily be envisaged by one skilled in the art.

The Nested PCR is a two-step PCR reaction using internal primers within the primary PCR in order to improve the sensitivity of PCR. Firstly, a primary PCR reaction is carried out using primary primers and template DNA from a sample. A secondary PCR reaction with the reaction products and inner primers provides a method of gene amplification that improves detection sensitivity, and is described in detail at p 65 of PCR Primer, A Laboratory Manual, 2^{nd} edition.

The micro-PCR which uses a small chip for PCR is able to formulate 20 μl of reaction composition containing only template DNA and inner primers on the chip, from which 1 μl is taken and injected into the silicon chip. The method has the advantages of using a small amount of reaction composition and monitoring ease. The chip is mounted on a TMC-1000 module and the DNA amplification is monitored in real-time during the PCR reaction. Micro PCR TMC-1000™ (a PCR device manufactured by Samsung Techwin) is an example of a commercially available device.

The PCR can be done using a common thermal block PCR or micro-PCR device, but is not limited thereto.

The detection method of this invention can be used for any material in which the aforementioned pathogens may be discovered, and is preferably used for foods or feeds. For instance, a sample suspected of being contaminated by a pathogen is suspended in sterile water or 0.85% physiological saline, and then treated with Protease K. After the pellet is obtained it is heat extracted to prepare a PCR sample, then undergoes PCR. The PCR result obtained is analyzed by a general method of detection for PCR, such as electrophoresis. The heat extraction is a general method of DNA separation, where for example, the pellet is suspended in sterile water, and treated with phenol/chloroform (1:1 volume mixture) from which the upper layer is obtained and mixed with ethanol, then centrifuged to obtain the DNA pellet. Detailed reaction conditions and time follow usual practices.

The method of the present invention can specifically and efficiently detect pathogens, and nested PCR significantly elevates the detection limit of pathogens. Notably, it elevates the existing detection limit 10⁵ CFU/ml to 100˜10 CFU/ml.

In addition, the present invention provides a pathogen detection kit comprising a PCR primer pair for detecting one or more pathogens. A pathogen detection kit comprises the usual components (reacting buffer, Taq DNA polymerase, labeling material, etc.) of a PCR microbial detection kit, and includes seven pairs of primers (SEQ ID NO:3-4, 7-8, 11-14, 17-18, 21-24).

In a preferred embodiment of the present invention, the detection kit comprises:

(1) a primer set comprising one or more pairs of primer selected from the group consisting of the primers shown in SEQ ID NO:3-4, SEQ ID NO:7-8, SEQ ID NO:11-14, SEQ ID NO:17-18, and SEQ ID NO:21-24,

(2) Reaction Buffer, and

(3) Taq DNA polymerase.

The detection kit also comprises a further one or more pair of primers selected from the group consisting of the primers shown in SEQ ID NO:1-2, SEQ ID NO: 5-6, SEQ ID NO: 9-10, SEQ ID NO:15-16 and SEQ ID NO:19-20,

In a preferred embodiment of the present invention, the detection kit comprises:

(1) a primer set comprising one or more primer pairs selected from the group consisting of primers shown in SEQ ID NO:3-4, SEQ ID NO:7-8, SEQ ID NO:11-14, SEQ ID NO:17-18, SEQ ID NO: 21-24,

(2) 4× Greenstar PCR Master Mix including Taq DNA polymerase and SYBR Green

(3) control DNA, and

(4) 10 mM MgCl₂

### MODE FOR INVENTION

The present invention is further explained in more detail with reference to the following examples. These examples, however, should not be interpreted as limiting the scope of the present invention in any manner.

### EXAMPLE 1

### Design and Preparation of PCR Primers for Amplifying Specific DNA of Five Kinds of Pathogens

To simultaneously detect Salmonella spp., Staphylococcus aureus, E. coli O157:H7, Listeria monocytogenes, and Vibrio parahaemolyticus, the primers listed in Table 2 above were designed and synthesized.

### EXAMPLE 2

### Nested PCR

2-1: Salmonella spp.

20 ul of Proteinase K (20 mg/mL) was added to 1 ml of Salmonella enteritidis (KCCM12021) of 108 to 1 CFU/ml, and then reacted for 10 minutes at 60° C. After finishing the reaction, the pellet was obtained by centrifuging the reaction product at 10,000 g rpm for 5 minutes, and suspended in 200 uL of sterilized water. This was heated at 105° C. for 20 minutes, then mixed with an equal volume of phenol/chloroform, and centrifuged at 10,000 g rpm, for 5 minutes to obtain the supernatant. The supernatant was mixed with an equal volume of ethanol and centrifuged at 10,000 g rpm for 5 minutes to recover the pellet, then 20 uL of distilled water was added to the pellet to prepare the PCR sample.

The PCR was performed in two groups. That is, PCR using the primer pair shown in SEQ ID NO: 1 and 2 was performed for one group, while for the other group, the reaction product from PCR with the primer pair shown in SEQ ID NOs:1 and 2 was used to perform a second round of PCR with the added primer pair shown in SEQ ID NOs: 3 and 4. The PCR reaction conditions of two groups were the same in other respects and were as follows.

<PCR Conditions>

94° C., 5 minutes—>(94° C., 30 seconds—>60° C., 30 seconds—>72° C., 30 seconds), 35 cycles—>72° C., 5 minutes.

TABLE 3
PCR reactant composition

| | 1^{st} PCR composition | | 2^{nd} PCR composition | |
|---|---|---|---|---|
| 5X Reaction Buffer | 5 | μl | 5 | μl |
| Primer 1 | 4 | μl SEQ ID NO: 1 | 4 | μl SEQ ID NO: 3 |
| Primer 2 | 4 | μl SEQ ID NO: 2 | 4 | μl SEQ ID NO: 4 |
| Taq Polymerase | 1 | μl | 1 | μl |
| (1 U/μl) | | | | |
| Template DNA | <50 | ng | <50 | ng |
| Total Volume | 25 | μl | 25 | μl |

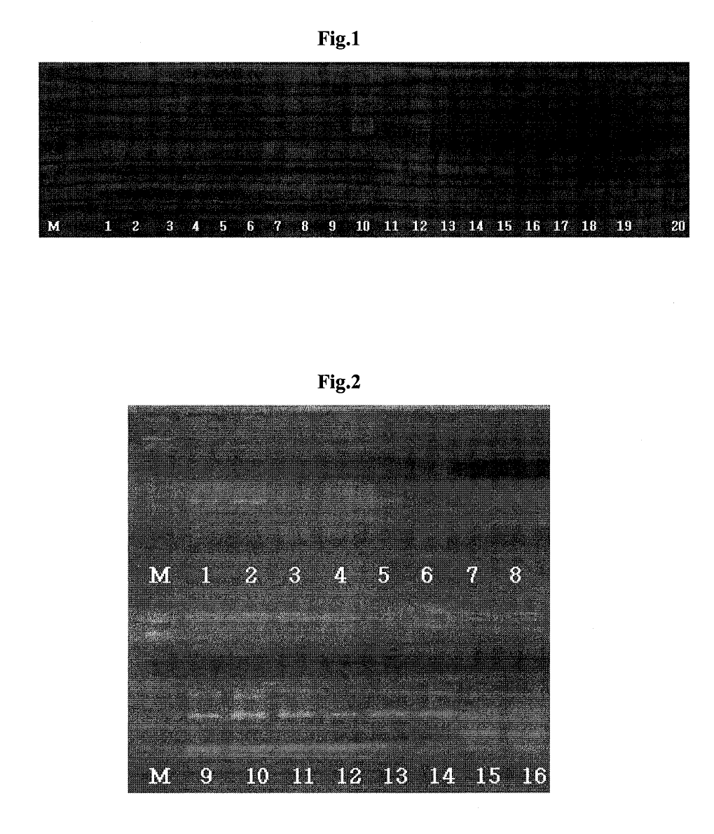

FIG. 1 shows the PCR result for Salmonella enteritidis. Lanes 1 to 10 correspond to the primer set shown in SEQ ID NO:1 and 2 and Lanes 11 to 20 correspond to nested PCR using two pairs of primers shown in SEQ ID NO:1 to 4. The concentration of the Salmonella enteritidis in each lane is as follows:

| | |
|---|---|
| Lanes 1 and 11: 10⁸ CFU/ml | Lanes 2 and 12: 10⁷ CFU/ml |
| Lanes 3 and 13: 10⁶ CFU/ml | Lanes 4 and 14: 10⁵ CFU/ml |
| Lanes 5 and 15: 10⁴ CFU/ml | Lanes 6 and 16: 10³ CFU/ml |
| Lanes 7 and 17: 10² CFU/ml | Lanes 8 and 18: 10 CFU/ml |
| Lanes 9 and 19: 1 CFU/ml | Lanes 10 and 20: Salmonella DNA 10 ng. |

The nested PCR of FIG. 1 shows amplification products of 678 bp and 200 bp, and the improved detection limit compared with the PCR result using the primer set shown in SEQ ID NO:1 and 2 is notable. That is, for the result of PCR using the primer pair shown in SEQ ID NO:1 and 2 for Salmonella enteritidis the 678 bp amplification product can only be confirmed at 10⁸ CFU/ml, while in the nested PCR using a pair of primers shown in SEQ ID NO:3 and 4 to further amplify the PCR product from reaction with the primer pair shown in SEQ ID NO:1 and 2 resulted in being able to detect the 200 bp amplification product at 10 CFU/ml.

2-2. Staphylococcus aureus

10⁷ to 1 CFU/ml of Staphylococcus aureus (KCCM1927) was prepared, and then DNA was extracted in the same way as in 2-1.

PCR was performed in two groups. That is, PCR using the primer pair shown in SEQ ID NO:5 and 6 was performed for one group. For the other group, the reaction was carried out with all the primers shown in SEQ ID NO:5 to 8 mixed in with the reactant composition. The PCR conditions of two group PCR were otherwise same and the PCR reaction conditions and composition were as in 2-1.

FIG. 2 shows the PCR result for Staphylococcus aureus. Lanes 1 to 8 correspond to the primer set shown in SEQ ID NO:5 and 6 and Lanes 9 to 16 correspond to nested PCR using two pairs of primers shown in SEQ ID NO:5 to 8. The concentration of the Staphylococcus aureus in each lane is as follows:

Lanes 1 and 9: 10⁷ CFU/ml

Lanes 2 and 10: 10⁶ CFU/ml

Lanes 3 and 11: 10⁵ CFU/ml

Lanes 4 and 12: 10⁴ CFU/ml

Lanes 5 and 13: 10³ CFU/ml

Lanes 6 and 14: 10² CFU/ml

Lanes 7 and 15: 10 CFU/ml

Lanes 8 and 16: 1 CFU/ml

FIG. 2 shows that the detection limit of PCR with the primer pair shown in SEQ ID NO:5 to 6 is was about 10⁶ CFU/ml, while the detection limit is significantly improved to 10² CFU/ml when a further primer pair shown in SEQ ID NO:7 and 8 is used in nested PCR. In lanes 15 and 16, only RNA bands were detected.

2-3. E. coli O157

4×10⁸ to 1 CFU/ml of E. coli O157:H7 ATCC12024 was prepared, and then DNA was extracted in the same way as in 2-1.

The PCR was performed in two groups. That is, PCR using the primer pair shown in SEQ ID NO:9 and 10 was performed for one group. For the other group, nested PCR using the primer pairs shown in SEQ ID NO:9 to 12 or SEQ ID NO:9-10 and 13-14 were performed. The reaction conditions of two groups PCR were otherwise the same. PCR reaction conditions and reaction composition were the same as in 2-1.

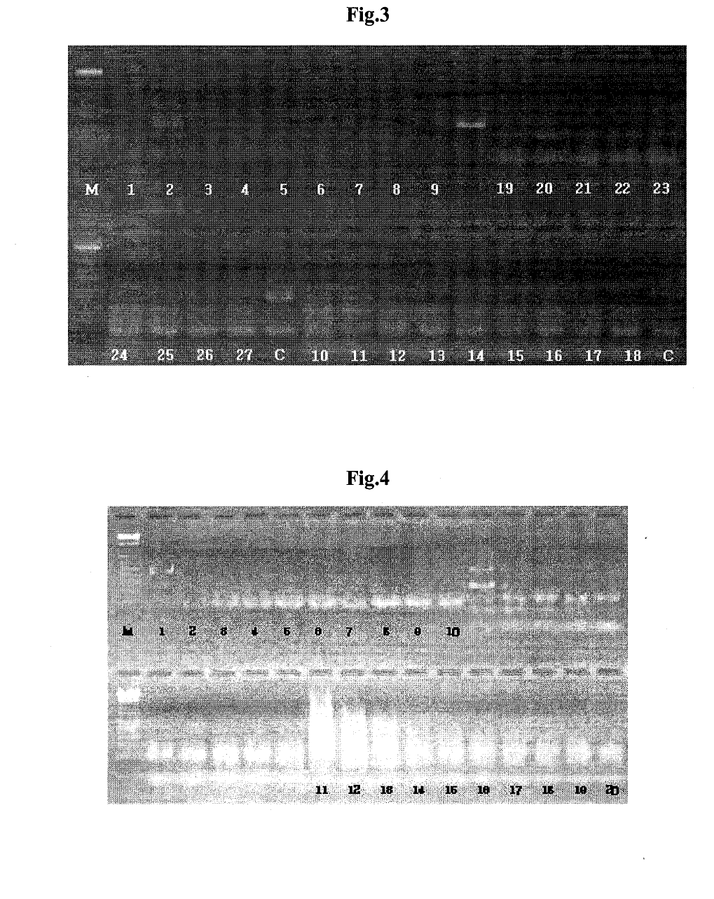

The PCR result for E. coli O157:H7 is shown in FIG. 3.

The PCR result of primer set shown in SEQ ID NO:9 and 10 is shown in Lanes 1 to 9, and nested PCR using two primer pairs shown in SEQ ID NO:9 to 12 are shown in Lanes 9 to 18, and nested PCR using two primer pairs shown in SEQ ID NO:9-10 and 13-14 are shown in Lanes 19 to 27. The concentration of the E. coli O157:H7 is as follows:

Lanes 1, 10 and 19: 4×10⁸ CFU/ml

Lanes 2, 11 and 20: 4×10⁷ CFU/ml

Lanes 3, 12 and 21: 4×10⁶ CFU/ml

Lanes 4, 13 and 22: 4×10⁵ CFU/ml

Lanes 5, 14 and 23: 4×10⁴ CFU/ml

Lanes 6, 15 and 24: 4×10³ CFU/ml

Lanes 7, 16 and 25: 4×100 CFU/ml

Lanes 8, 17 and 26: 4×10 CFU/ml

Lanes 9, 18 and 27: 4×1 CFU/ml

FIG. 3 shows that when PCR is carried out using the primer pair shown in SEQ ID NO:9 and 10 (lane 1 to 9), a 208 bp DNA band in Lane 1 confirms that the detection limit is about 10⁸ CFU/ml. On the other hand, nested PCR using the primer pair shown in SEQ ID NO:11 and 12 (Lanes 10 to 18) detects a 129 bp DNA band in Lanes 10 to 17. So the detection limit is remarkably improved. Nested PCR using the primer pair shown in SEQ ID NO:13 and 14 (Lanes 19 to 27) detects a 108 bp DNA band in Lanes 19 to 25, and indicates that the detection limit is 10² CFU/ml.

2-4. Listeria monocytogenes

1.1×10⁹ to 1 CFU/ml of Listeria monocytogenes ATCC 19112 was prepared, and then DNA was extracted in the same way as in 2-1.

The PCR was performed in two groups. That is, PCR using the primer pair shown in SEQ ID NO:15 and 16 was performed for one group. For the other group, nested PCR using the primer pair shown in SEQ ID NO:17 and 18 was performed. The reaction conditions of two groups were otherwise the same. PCR reaction conditions and composition were the same as in 2-1.

The PCR result for Listeria monocytogenes is shown in FIG. 4.

The PCR result of primer set shown in SEQ ID NO:15 and 16 is shown in Lanes 1 to 10, and the nested PCR of the primer pair shown in SEQ ID NO:17 and 18 is shown in Lanes 11 to 20. The concentration of the Listeria monocytogenes is as follows:

Lanes 1 and 11:Listeria monocytogenes 1.1×10⁹ CFU/ml

Lanes 2 and 12: 1.1×10⁸ CFU/ml

Lanes 3 and 13: 1.1×10⁷ CFU/ml

Lanes 4 and 14: 1.1×10⁶ CFU/ml

Lanes 5 and 15: 1.1×10⁵ CFU/ml

Lanes 6 and 16: 1.1×10⁴ CFU/ml

Lanes 7 and 17: 1.1×10³ CFU/ml

Lanes 8 and 18: 1.1×100 CFU/ml

Lanes 9 and 19: 1.1×10 CFU/ml

Lanes 10 and 20: 1.1×1 CFU/ml.

In FIG. 4, PCR using the primer pair shown in SEQ ID NO:15 and 16 (Lane 1 to 10) detected an amplified product of 454 bp, and nested PCR using a further primer pair shown in SEQ ID NO:17 and 18 (Lanes 11 to 20) detected amplified products of 454 bp and 191 bp. However nested PCR was more useful for the detection of lower concentrations of Listeria monocytogenes.

2-5. Vibrio parahaemoliticus

2.1×10¹⁰ to 1 CFU/ml of Vibrio parahaemoliticus KCCM11965 was prepared, and then DNA was extracted in the same way as in 2-1.

The PCR was performed in two groups. That is, PCR using the primer pair shown in SEQ ID NO:19 and 20 was performed for one group. For the other group, nested PCR using the primer pairs shown in SEQ ID NO:19 to 22 or SEQ ID NO:19-20 and 23-24 was performed. The reaction conditions of two groups were otherwise the same. PCR reaction conditions and reaction composition were the same as in 2-1.

The PCR result for Vibrio parahaemoliticus is shown in FIG. 5.

The PCR result using the primer set shown in SEQ ID NO:19 and 20 is shown in Lanes 1 to 11, and the results of nested PCR using two pairs of primers shown in SEQ ID NO:19 are shown in Lanes 12 to 22, and that of nested PCR using two pairs of primers shown in SEQ ID NO: 19-20 and 23-24 are shown in Lanes 23 to 33. The concentration of the Vibrio parahaemoliticus is as follows:

Lanes 1, 12 and 23: Vibrio parahaemoliticus 2.1×10¹⁰ CFU/ml

Lanes 2, 13 and 24: 2.1×10⁹ CFU/ml

Lanes 3, 14 and 25: 2.1×10⁸ CFU/ml

Lanes 4, 15 and 26: 2.1×10⁷ CFU/ml

Lanes 5, 16 and 27: 2.1×10⁶ CFU/ml

Lanes 6, 17 and 28: 2.1×10⁵ CFU/ml

Lanes 7, 18 and 29: 2.1×10⁴ CFU/ml

Lanes 8, 19 and 30: 2.1×10³ CFU/ml

Lanes 9, 20 and 31: 2.1×100 CFU/ml

Lanes 10, 21 and 32: 2.1×10 CFU/ml

Lanes 11, 22 and 33: 2.1×1 CFU/ml.

In FIG. 5, PCR using the primer pair shown in SEQ ID NO:19 and 20 for Vibrio parahaemoliticus KCCM11965 shows an amplification product of 375 bp in only lanes 1 to 3. That shows that the detection limit is 10⁸ CFU/ml in case of PCR using the primer pair shown in SEQ ID NO:19 and 20. The result of nested PCR using a further primer pair shown in SEQ ID NO:21 and 22 with the amplification product from using the primer pair shown in SEQ ID NO:19 and 20 showed a 219 bp amplification product in lanes 12 to 22. Furthermore, nested PCR using a further primer pair shown in SEQ ID NO: 23 and 24 detected a 153 bp amplification product in lanes 23 to 33

Accordingly, nested PCR using the primer pairs of the present invention can detect pathogens at 10 to 1 CFU/ml concentrations, which is a remarkably superior detection limit.

### EXAMPLE 3

### Verification of Primers

To verify the pathogen specificity of the primers in Example 1, PCR was performed on DNA of microorganism similar to the pathogens.

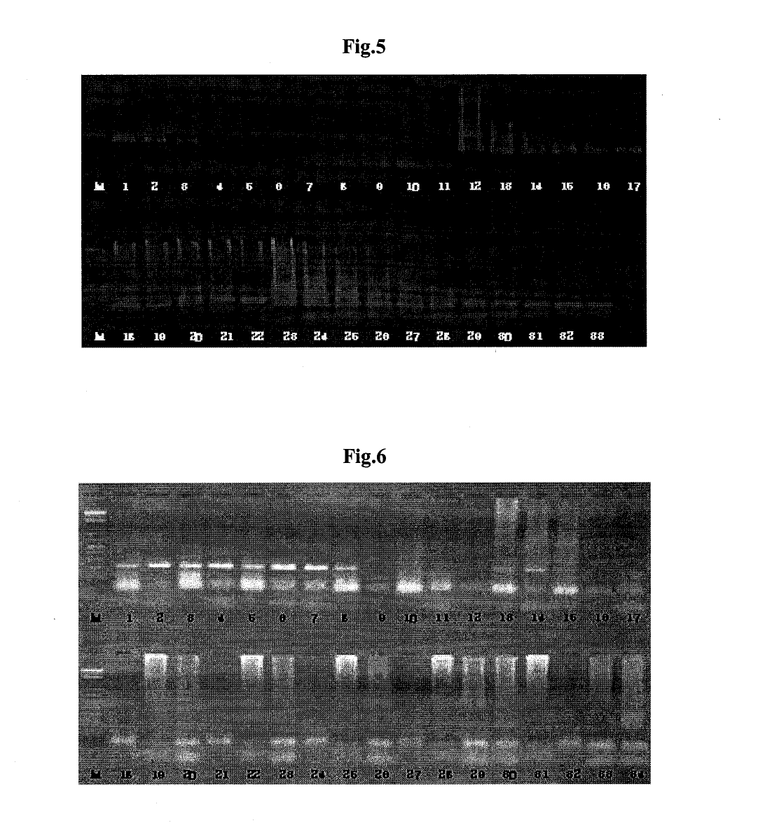

3-1. The Primer Pair Shown in SEQ ID NO:3 and 4
FIG. 6 is a result obtained by performing nested PCR with primers shown in SEQ ID NO:3 and 4 for detecting Salmonella spp., and the lanes are described below, where lane M is a size marker.

A 200 bp PCR product is confirmed in Lanes 1-8, 13-14, 30 and 34 of FIG. 6. Therefore it can be seen that the primer pair shown in SEQ ID NO:3 and 4 can specifically detect Salmonella spp

TABLE 4

| Lane | Pathogen | Concentration | Primer |
|---|---|---|---|
| 1 | Salmonella enterifidis KCCM12021 | 10² CFU/ml | SEQ ID NO: 3 and 4 |
| 2 | Salmonella enterifidis KCCM12021 | 10 CFU/ml | SEQ ID NO: 3 and 4 |
| 3 | Salmonella choleraesuis subsp cholerae | 10² CFU/ml | SEQ ID NO: 3 and 4 |
| | KCCM41035 | | |
| 4 | Salmonella choleraesuis subsp cholerae | 10 CFU/ml | SEQ ID NO: 3 and 4 |
| | KCCM41035 | | |
| 5 | Salmonella choleraesuis KCCM41575 | 10² CFU/ml | SEQ ID NO: 3 and 4 |
| 6 | Salmonella choleraesuis KCCM41575 | 10 CFU/ml | SEQ ID NO: 3 and 4 |
| 7 | Salmonella bongori KCCM41758 | 10² CFU/ml | SEQ ID NO: 3 and 4 |
| 8 | Salmonella bongori KCCM41758 | 10 CFU/ml | SEQ ID NO: 3 and 4 |
| 9 | Yersinia enterocolitica KCCM41675 | 10⁸ CFU/ml | SEQ ID NO: 1 and 2 |
| 10 | Yersinia enterocolitica KCCM41675 | 10⁸ CFU/ml | SEQ ID NO: 3 and 4 |
| 11 | Yersinia enterocolitica KCCM41675 | 10⁷ CFU/ml | SEQ ID NO: 3 and 4 |
| 12 | Shigella boydii KCCM41646 | 10⁸ CFU/ml | SEQ ID NO: 1 and 2 |
| 13 | Salmonella typhimurium ATCC14023 | 10⁸ CFU/ml | SEQ ID NO: 3 and 4 |
| 14 | Salmonella typhimurium ATCC14023 | 10⁷ CFU/ml | SEQ ID NO: 3 and 4 |
| 15 | Citrobacter freund KCCM11931 | 10⁸ CFU/ml | SEQ ID NO: 1 and 2 |
| 16 | Citrobacter freund KCCM11931 | 10⁸ CFU/ml | SEQ ID NO: 3 and 4 |
| 17 | Citrobacter freund KCCM11931 | 10⁷ CFU/ml | SEQ ID NO: 3 and 4 |
| 18 | Escherichia coli KCTC1467 | 10⁸ CFU/ml | SEQ ID NO: 1 and 2 |
| 19 | Escherichia coli KCTC1467 | 10⁸ CFU/ml | SEQ ID NO: 3 and 4 |
| 20 | Escherichia coli KCTC1467 | 10⁷ CFU/ml | SEQ ID NO: 3 and 4 |
| 21 | Cytrobacter freudii KCTC2006 | 10⁸ CFU/ml | SEQ ID NO: 1 and 2 |
| 22 | Cytrobacter freudii KCTC2006 | 10⁸ CFU/ml | SEQ ID NO: 3 and 4 |
| 23 | Cytrobacter freudii KCTC2006 | 10⁷ CFU/ml | SEQ ID NO: 3 and 4 |
| 24 | Shigella sonnei KCTC2518 | 10⁸ CFU/ml | SEQ ID NO: 1 and 2 |
| 25 | Shigella sonnei KCTC2518 | 10⁸ CFU/ml | SEQ ID NO: 3 and 4 |
| 26 | Shigella sonnei KCTC2518 | 10⁷ CFU/ml | SEQ ID NO: 3 and 4 |
| 27 | Shigella flexneri KCTC2517 | 10⁸ CFU/ml | SEQ ID NO: 1 and 2 |
| 28 | Shigella flexneri KCTC2517 | 10⁸ CFU/ml | SEQ ID NO: 3 and 4 |
| 29 | Shigella flexneri KCTC2517 | 10⁷ CFU/ml | SEQ ID NO: 3 and 4 |
| 30 | Salmonella cholerae KCTC2931 | 10² CFU/ml | SEQ ID NO: 3 and 4 |
| 31 | Shigella sonnei KCTC2009 | 10⁸ CFU/ml | SEQ ID NO: 1 and 2 |
| 32 | Shigella sonnei KCTC2009 | 10⁸ CFU/ml | SEQ ID NO: 3 and 4 |
| 33 | Shigella sonnei KCTC2009 | 10⁷ CFU/ml | SEQ ID NO: 3 and 4 |
| 34 | Salmonella cholelaesuis KCTC2929 | 10² CFU/ml | SEQ ID NO: 3 and 4 |

3-2. The Primer Pair Shown in SEQ ID NO:7 and 8

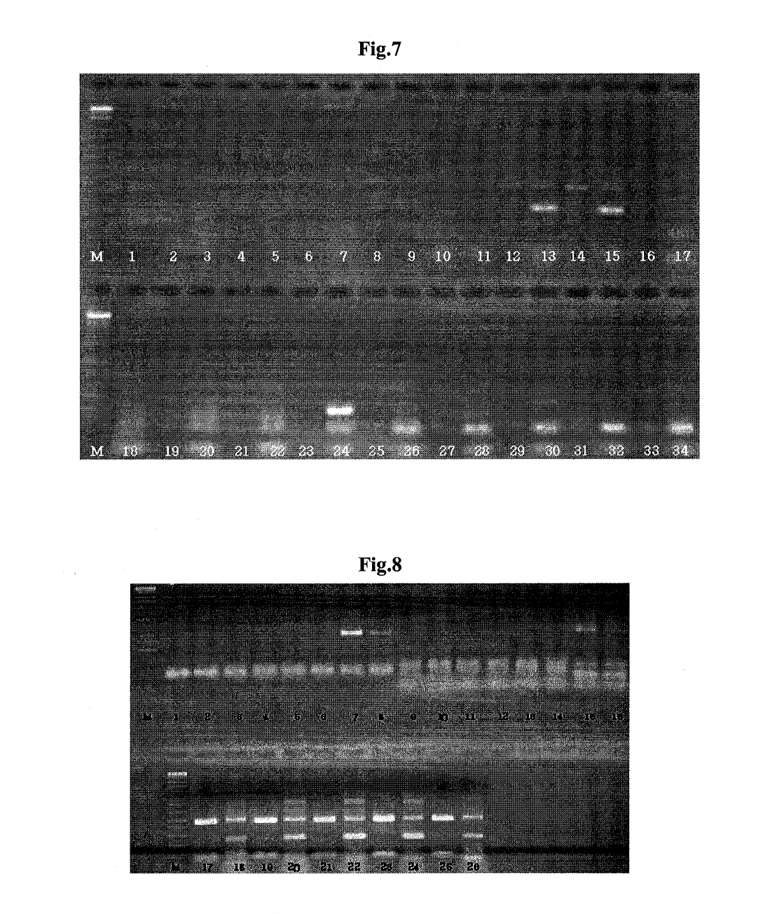

FIG. 7 is a result obtained by performing nested PCR with primers shown in SEQ ID NO:7 and 8 for detecting Staphylococcus aureus, and the lanes are described below, where lane M is a size marker.

| Lane | Pathogen | Concentration | Primer |
|---|---|---|---|
| 1 | Staphylococcus, ylous KCCM41465 | 10⁸ CFU/ml | SEQ ID NO: 5 and |
| 2 | Staphylococcus, ylous KCCM41465 | 10⁸ CFU/ml | SEQ ID NO: 7 and |
| 3 | Staphylococcus, ylous KCCM11764 | 10² CFU/ml | SEQ ID NO: 7 and |
| 4 | Staphylococcus arlettae KCTC3588 | 10⁸ CFU/ml | SEQ ID NO: 7 and |
| 5 | Staphylococcus arlettae KCTC3588 | 10⁸ CFU/ml | SEQ ID NO: 7 and |
| 6 | Bacillus licheniformis KCTC1831 | 10⁸ CFU/ml | SEQ ID NO: 5 and |
| 7 | Bacillus licheniformis KCTC1831 | 10⁸ CFU/ml | SEQ ID NO: 7 and |
| 8 | Bacillus licheniformis KCTC3006 | 10⁸ CFU/ml | SEQ ID NO: 7 and |
| 9 | Bacillus licheniformis KCTC3006 | 10⁸ CFU/ml | SEQ ID NO: 7 and |
| 10 | Bacillus cereus KCTC1526 | 10⁸ CFU/ml | SEQ ID NO: 5 and |
| 11 | Bacillus cereus KCTC1526 | 10⁸ CFU/ml | SEQ ID NO: 7 and |
| 12 | Staphylococcus aureus KCTC1916 | 10² CFU/ml | SEQ ID NO: 5 and |
| 13 | Staphylococcus aureus KCTC1928 | 10⁵ CFU/ml | SEQ ID NO: 7 and |
| 14 | Staphylococcus aureus KCTC1928 | 10⁵ CFU/ml | SEQ ID NO: 5 and |
| 15 | Staphylococcus aureus KCTC1927 | 10⁵ CFU/ml | SEQ ID NO: 7 and |
| 16 | Staphylococcus aureus KCTC1927 | 10⁵ CFU/ml | SEQ ID NO: 7 and |
| 17 | Bacillus subtilis KCTC3013 | 10⁸ CFU/ml | SEQ ID NO: 5 and |
| 18 | Bacillus subtilis KCTC3013 | 10⁸ CFU/ml | SEQ ID NO: 7 and |
| 19 | Bacillus subtilis KCTC1661 | 10⁸ CFU/ml | SEQ ID NO: 5 and |
| 20 | Bacillus subtilis KCTC1661 | 10⁸ CFU/ml | SEQ ID NO: 7 and |
| 21 | Staphylococcus lentus KCTC3577 | 10⁸ CFU/ml | SEQ ID NO: 5 and |
| 22 | Staphylococcus lentus KCTC3577 | 10⁸ CFU/ml | SEQ ID NO: 7 and |
| 23 | Staphylococcus epidermidis KCTC1917 | 10⁸ CFU/ml | SEQ ID NO: 5 and |
| 24 | Staphylococcus epidermidis KCTC1917 | 10⁸ CFU/ml | SEQ ID NO: 7 and |
| 25 | Staphylococcus cohnii KCTC3574 | 10⁸ CFU/ml | SEQ ID NO: 5 and |
| 26 | Staphylococcus cohnii KCTC3574 | 10⁸ CFU/ml | SEQ ID NO: 7 and |
| 27 | Bacillus cereus KCTC3674 | 10⁸ CFU/ml | SEQ ID NO: 5 and |
| 28 | Bacillus cereus KCTC3674 | 10⁸ CFU/ml | SEQ ID NO: 7 and |
| 29 | Bacillus subtilis KCTC2213 | 10⁸ CFU/ml | SEQ ID NO: 5 and |
| 30 | Bacillus subtilis KCTC2213 | 10⁸ CFU/ml | SEQ ID NO: 7 and |
| 31 | Staphylococcus caprae KCTC3583 | 10⁸ CFU/ml | SEQ ID NO: 5 and |
| 32 | Staphylococcus caprae KCTC3583 | 10⁸ CFU/ml | SEQ ID NO: 7 and |
| 33 | Staphylococcus warneri KCTC3340 | 10⁸ CFU/ml | SEQ ID NO: 5 and |
| 34 | Staphylococcus warneri KCTC3340 | 10⁸ CFU/ml | SEQ ID NO: 7 and |

Staphylococcus aureus in lanes 12 to 15 is the only ones detected in FIG. 7, it means the primer pair shown in SEQ ID NO: 7 and 8 can specifically detect Staphylococcus aureus.

3-3. The Primer Pair Shown in SEQ ID NO:17 and 18

FIG. 8 is a result obtained by performing nested PCR with primers shown in SEQ ID NO: 17 and 18 for detecting Listeria monocytogenes, and the lanes are described below, where lane M is a size marker.

FIG. 8 shows 454 bp and/or 191 bp products amplified by SEQ ID NO:17 and 18 in lanes 17 to 26.

TABLE 6

| Lane | Pathogen | Concentration | Primer |
|---|---|---|---|
| 1 | Listeria grayi ATCC25400 | 10⁸ CFU/ml | SEQ ID NO: 15 and |
| | | | 16 |
| 2 | Listeria murra ATCC25402 | 10⁸ CFU/ml | SEQ ID NO: 15 and |
| | | | 16 |
| 3 | Listeria murra ATCC25403 | 10⁸ CFU/ml | SEQ ID NO: 15 and |
| | | | 16 |
| 4 | Listeria grayi ATCC700545 | 10⁸ CFU/ml | SEQ ID NO: 15 and |
| | | | 16 |
| 5 | Listeria ivanovii ATCC49953 | 10⁸ CFU/ml | SEQ ID NO: 15 and |
| | | | 16 |
| 6 | Listeria ivanovii subsp. Londoniensis | 10⁸ CFU/ml | SEQ ID NO: 15 and |
| | ATCC49954 | | 16 |
| 7 | Listeria innocua ATCC33090 | 10⁸ CFU/ml | SEQ ID NO: 15 and |
| | | | 16 |
| 8 | Listeria innocua ATCC33091 | 10⁸ CFU/ml | SEQ ID NO: 15 and |
| | | | 16 |
| 9 | Listeria grayi ATCC25400 | 10⁸ CFU/ml | SEQ ID NO: 17 and |
| | | | 18 |
| 10 | Listeria murra ATCC25402 | 10⁸ CFU/ml | SEQ ID NO: 17 and |
| | | | 18 |
| 11 | Listeria murra ATCC25403 | 10⁸ CFU/ml | SEQ ID NO: 17 and |
| | | | 18 |
| 12 | Listeria grayi ATCC700545 | 10⁸ CFU/ml | SEQ ID NO: 17 and |
| | | | 18 |
| 13 | Listeria ivanovii ATCC49953 | 10⁸ CFU/ml | SEQ ID NO: 17 and |
| | | | 18 |
| 14 | Listeria ivanovii subsp. Londoniensis | 10⁸ CFU/ml | SEQ ID NO: 17 and |
| | ATCC49954 | | 18 |
| 15 | Listeria innocua ATCC33090 | 10⁸ CFU/ml | SEQ ID NO: 17 and |
| | | | 18 |
| 16 | Listeria innocua ATCC33091 | 10⁸ CFU/ml | SEQ ID NO: 17 and |
| | | | 18 |
| 17 | Listeria monocytogenes ATCC3152 | 10⁵ CFU/ml | SEQ ID NO: 15 and |
| | | | 16 |
| 18 | Listeria monocytogenes ATCC3152 | 10⁵ CFU/ml | SEQ ID NO: 17 and |
| | | | 18 |
| 19 | Listeria monocytogenes ATCC15313 | 10⁵ CFU/ml | SEQ ID NO: 15 and |
| | | | 16 |
| 20 | Listeria monocytogenes ATCC15313 | 10⁵ CFU/ml | SEQ ID NO: 17 and |
| | | | 18 |
| 21 | Listeria monocytogenes ATCC19112 | 10⁵ CFU/ml | SEQ ID NO: 15 and |
| | | | 16 |
| 22 | Listeria monocytogenes ATCC19112 | 10⁵ CFU/ml | SEQ ID NO: 17 and |
| | | | 18 |
| 23 | Listeria monocytogenes ATCC19113 | 10⁵ CFU/ml | SEQ ID NO: 15 and |
| | | | 16 |
| 24 | Listeria monocytogenes ATCC19113 | 10⁵ CFU/ml | SEQ ID NO: 17 and |
| | | | 18 |
| 25 | Listeria monocytogenes ATCC BAA-676 | 10⁵ CFU/ml | SEQ ID NO: 17 and |
| | | | 18 |
| 26 | Listeria monocytogenes ATCC BAA-676 | 10⁵ CFU/ml | SEQ ID NO: 17 and |
| | | | 18 |

### EXAMPLE 4

### PCR Detection Kit

TABLE 7

| Number | Detection kit | Composition |
|---|---|---|
| 1 | Detection kit for | 5X Reaction Buffer, Taq DNA Polymerase(1 U/μl) |
| | Salmonella spp. | Primer mixture solution (SEQ ID NO: 1 to 4) |
| | | Control group DNA(purified DNA of Salmonella spp., 10 ng/μl) |
| 2 | Detection kit for | 5X Reaction Buffer, Taq DNA Polymerase(1 U/μl) |
| | Staphylococcus aureus | Primer mixture solution(SEQ ID NO: 5 to 8) |
| | | Control group DNA(purified DNA of Staphylococcus aureus, |
| | | 10 ng/μl) |
| 3 | Detection kit for | 5X Reaction Buffer, Taq DNA Polymerase(1 U/μl) |
| | Escherichia coli | Primer mixture solution (SEQ ID NO: 9 to 12 or SEQ ID |
| | O157:H7 | NO: 9-10, 13-14) |
| | | Control group DNA(Purified DNA of E. coli O157:H7, 10 ng/μl) |
| 4 | Detection kit for | 5X Reaction Buffer, Taq DNA Polymerase(1 U/μl) |
| | Listeria monocytogenes | Primer mixture solution (SEQ ID NO: 15 to 18) |
| | | Control group DNA(Purified DNA of Listeria monocytogenes, |
| | | 10 ng/μl) |
| 5 | Detection kit for | 5X Reaction Buffer, Taq DNA Polymerase(1 U/μl) |
| | Vibrio | Primer mixture solution(SEQ ID NO: 19 to 22 or SEQ ID |
| | parahaemolyticus | NO: 19-20, 23-24) |
| | | Control group DNA(Purified DNA of Vibrio |
| | | parahaemolyticus, 10 ng/μl) |
| 6 | Mutiple detection kit | 5X Reaction Buffer, Taq DNA Polymerase(1 U/μl) |
| | | Primer mixture solution(SEQ ID NO: 1-12 and 15-22, or SEQ |
| | | ID NO: 1-10, 13-20 and 23-24) |
| | | Control group DNA(10 ng/μl) |
| | | The control DNA consists of DNA of Salmonlla spp. And |
| | | DNA of Staphylococcus aureous, DNA of E. coli |
| | | O157:H7, DNA of Listeria moncytogenes, and DNA of |
| | | Vibrio parahemoliticus. |

### EXAMPLE 5

### Detection for Food Borne Bacteria in Food

PCR was performed on culture solutions of intentionally contaminated foods by using the detection kit in example 4-1.

Each specimen (25 g) of four kinds of foods (sandwich, hamburger, sweet and sour pork, flying fish roe) was inoculated into LB (Luria Burtani) 225 ml and incubated at 37° C., 80 rpm for 16 hours. PCR using the primer pair shown in SEQ ID NO:1 and 2 was performed on 1 ml of culture solution to confirm the absence of Salmonella, then the remaining culture fluid was sterilized. 10⁸ CFU/ml of Salmonella typhimurium ATCC14023 was added to 9 ml of the sterilized culture fluid, and repeatedly diluted to make a 1 to 10⁷ CFU/ml specimen.

20 μl of Proteinase K (20 mg/ml) was added to 1 ml of the specimen, and then incubated at 60° C. for 10 minutes. The pellet was obtained by centrifuging the reaction product at 10,000 g rpm for 5 minutes, and suspended in 200 uL of sterilized water. This was heated at 105° C. for 20 minutes, then mixed with an equal volume of phenol/chloroform, and centrifuged at 10,000 g rpm, for 5 minutes to obtain the supernatant. The supernatant was mixed with an equal volume of ethanol and centrifuged at 10,000 g rpm for 5 minutes to recover the pellet, then 20 uL of distilled water was added to the pellet to prepare the PCR sample DNA.

The PCR was performed with Biometra T-personal Thermal PCR Machine.

<PCR Conditions>

94° C., 5 minutes—>primary (94° C., 30 seconds—>60° C., 150 seconds—>72° C., 30 seconds), 5 times cycles—>secondary (88° C., 30 seconds—>60° C., 30 seconds—>72° C., 30 seconds), 15 times cycles)—>tertiary (88° C., 30 seconds—>54° C., 30 seconds—>72° C., 30 seconds), 10 times cycles)—>quaternary (86° C., 30 seconds—>54° C., 30 seconds—>72° C., 30 seconds), 25 times—>72° C., 10 minutes

FIG. 9 is a result obtained by performing nested PCR with the two pairs of primers shown in SEQ ID NO: 1 to 4 on foods contaminated with Salmonella typhimurium ATCC14023, and the lanes are described below, where lane M is a size marker.

1:10 ng of Salmonella spp. DNA, PCR using the primer pair shown in SEQ ID NO: 1 and 2

2: Negative control group (not including DNA)

3: Salmonella typhimurium ATCC14023, 10⁵ CFU/ml—flying fish roe

4: Salmonella typhimurium ATCC14023, 10⁴ CFU/ml—flying fish roe

5: Salmonella typhimurium ATCC14023, 10³ CFU/ml—flying fish roe

6: Salmonella typhimurium ATCC14023, 10² CFU/ml—flying fish roe

7: Salmonella typhimurium ATCC14023, 10¹ CFU/ml—flying fish roe

8: Salmonella typhimurium ATCC14023, 10⁵ CFU/ml—sweet and sour pork

9: Salmonella typhimurium ATCC14023, 10⁴ CFU/ml—sweet and sour pork

10: Salmonella typhimurium ATCC14023, 10³ CFU/ml—sweet and sour pork

11: Salmonella typhimurium ATCC14023, 10² CFU/ml—sweet and sour pork

12: Salmonella typhimurium ATCC14023, 10¹ CFU/ml—sweet and sour pork

13: Salmonella typhimurium ATCC14023, 10⁴ CFU/ml—a hamburger

14: Salmonella typhimurium ATCC14023, 10³ CFU/ml—a hamburger

15: Salmonella typhimurium ATCC14023, 10² CFU/ml—a hamburger

16: Salmonella typhimurium ATCC14023, 10¹ CFU/ml—a hamburger

17: Salmonella typhimurium ATCC14023, 10⁵ CFU/ml—sandwich

18: Salmonella typhimurium ATCC14023, 10⁴ CFU/ml—sandwich

19: Salmonella typhimurium ATCC14023, 10³ CFU/ml—sandwich

20: Salmonella typhimurium ATCC14023, 10² CFU/ml—sandwich

21: Salmonella typhimurium ATCC14023, 10¹ CFU/ml—sandwich

In FIG. 9, lanes 3 to 21 show detection to 10 CFU/ml by amplification of a 200 bp DNA using the primer pair shown in SEQ ID NO:3 and 4.

### EXAMPLE 6

### MICRO-PCR

6-1. Micro-PCR Using a Pair of Primers in SEQ ID NO:3 to 4

Micro-PCR using TMC-1000™ (SAMSUNG Techwin) was performed on 10⁸ CFU/mL Salmonella enteritidis KCCM12021. The PCR was performed in two groups. That is, PCR using the primer pair shown in SEQ ID NO:1 and 2 was performed for one group. For the other group, two pairs of primers shown in SEQ ID NO:1 to 4 were used

Micro-PCR conditions were as below.

94° C., 10 minutes—>(94° C., 5 seconds—>60° C., 5 seconds—>72° C., 5 seconds), 100 times cycles—>72° C., 5 minutes

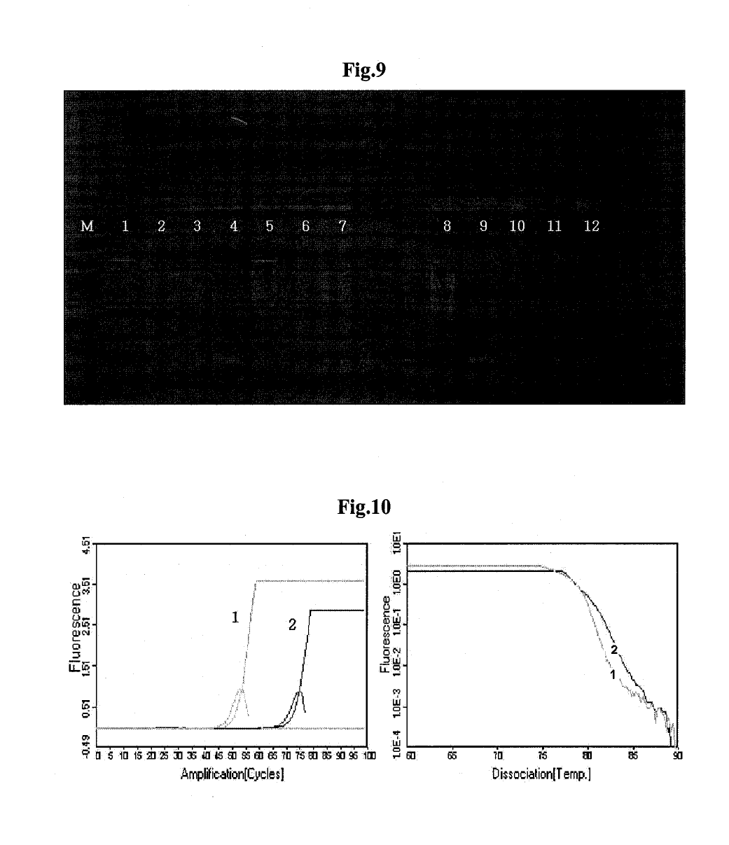

FIG. 10 is a result obtained by performing micro-PCR on Salmonella enteritidis KCCM12021.1 is where two pairs of primers shown in SEQ ID NO: 1 to 4 was used, and 2 is where the primer pair shown in SEQ ID NO:1 and 2 was used. The right-hand graph is a melting curve from which the Tm value can be obtained. Tm is the temperate at which double-stranded and single-stranded DNA each make up 50%. An average of the signal at this point can make up the standard quantitative curve.

As the CT (Cycles Time) value of 1 in FIG. 10 is quicker than that of line 2, it can be seen that PCR using a pair of primers shown in SEQ ID NO:3 and 4 is efficient.

### EXAMPLE 7

### Micro-PCR Detection Kit

7-1. Detection Kit Comprising Two Kinds of Primer Pairs

| | |
|---|---|
| 4X Greenstar | 5 μl |
| 10 mM MgCl₂ | 1 μl |
| Primer mixture solution (each 0.2 uM/μl) | 8 μl |
| Sample DNA (<500 ng) | 6 μl |
| Total Volume | 20 μl |

7-2. Detection Kit Comprising One (1) Kind of Primer Pair

| | | |
|---|---|---|
| 4X Greenstar | 5 | μl |
| 10 mM MgCl₂ | 1 | μl |
| Primer mixture solution (0.2 uM/μl) | 4 | μl |
| Sample DNA (<500 ng) | | |
| Nuclease-free water | upto 20 | μl |

The primer mixture solution optionally consists of Table 8 and Table 9

TABLE 8
A kit including two pairs of primers

| Detected Pathogen | Primer |
|---|---|
| Salmonella spp. | SEQ ID NO: 1, 2, 3 and 4 |
| Staphylococcus | SEQ ID NO: 5, 6, 7 and 8 |
| aureus | |
| E. coli O157 | SEQ ID NO: 9, 10, 11, and 12; or SEQ ID NO: 9, 10, 13 and 14 |
| Listeria monocytogenes | SEQ ID NO: 15, 16, 17 and 18 |
| Vibrio | SEQ ID NO: 19, 20, 21 and 22; or SEQ ID NO: 19, 20, 23 and 24 |
| parahaemolyticus | |
| Five kinds of pathogens | SEQ ID NO: 1 to 12, and 15 to 22; SEQ ID NO: 1 to 10, and 13 to |
| | 22; SEQ ID NO: 1 to 12, 15 to 20, and 23 to 24; or SEQ ID NO: 1 to |
| | 10, 13 to 20, and 23 to 24 |

TABLE 9
A kit including one pair of primers

| Detected pathogen | Primer |
|---|---|
| Salmonella spp. | SEQ ID NO: 3 and 4 |
| Staphylococcus aureus | SEQ ID NO: 7 and 8 |
| E. coli O157 | SEQ ID NO: 11 and 12; or SEQ ID NO: 13 |
| | and 14 |
| Listeria monocytogenes | SEQ ID NO: 17 and 18 |
| Vibrio parahaemolyticus | SEQ ID NO: 21 and 22; or SEQ ID NO: |
| | 23 and 24 |
| Five kinds of pathogens | SEQ ID NO: 3, 4, 7, 8, 11, 12, 17, 18, 21 |
| | and 22; |
| | SEQ ID NO: 3, 4, 7, 8, 13, 14, 17, 18, 21 |
| | and 22; |
| | SEQ ID NO: 3, 4, 7, 8, 11, 12, 17, 18, 23 and |
| | 24; or |
| | SEQ ID NO: 3, 4, 7, 8, 13, 14, 17, 18, 23 |
| | and 24 |

### EXAMPLE 8

### Detection of a Pathogen Causing Food Poisoning Using the Micro-PCR Detection Kit

8-1. Preparation of Sample

25 g of food suspected of being contaminated by Salmonella was measured and 100 ml of sterilized water was added then pulverized by a powdering machine. The pulverized sample was filtered through filter paper to obtain the filtrate, and then 20 μl of Proteinase K was added to 1 ml of the filtrate (20 mg/ml), and then incubated at 60° C. for 10 minutes. The pellet was obtained by centrifuging the reaction product at 10,000 g rpm for 5 minutes, and suspended in 200 uL of sterilized water. This was heated at 105° C. for 20 minutes, then mixed with an equal volume of phenol/chloroform, and centrifuged at 10,000 g rpm, for 5 minutes to obtain the supernatant. The supernatant was mixed with an equal volume of ethanol and centrifuged at 10,000 g rpm for 5 minutes to recover the pellet, then 20 uL of distilled water was added to the pellet to prepare the PCR sample DNA.

8-2. Micro-PCR Conditions

PCR reaction mixture was made by using the detection kit composition mentioned in Example 7, and then 1 μl of the mixture was injected without forming bubbles and sealed into a Genespector Micro chip, made by SAMSUNG ADVANCED INSTITUTE OF TECHNOLOGY.

The PCR was run on the chip as follows using a Genespector Micro PCR machine.

(1) Nested PCR using a two kinds of primer sets for one (1) kind of pathogen

TABLE 10
PCR condition

| | Reaction Conditions | Cycles |
|---|---|---|
| Primary reaction | Initial denaturation(94° C., 10 min.) -> Denaturation(94° C., 5 sec.), | 20 |
| condition | annealing(60° C., 5 sec.), extension(72° C., 5 sec.) | |
| Secondary | annealing(72° C., 5 sec.) -> extension(73° C., 5 sec.) | 30 |
| reaction condition | | |
| Tertiary reaction | denaturation(88° C., 5 sec.) -> annealing(54° C., 5 sec.) -> | 20 |
| condition | extension(72° C., 5 sec.) | |
| Quarternary | denaturation(72° C., 5 sec.) -> annealing(55° C., 5 sec.) -> | 50 |
| reaction condition | extension(73° C., 5 sec.) | |

(2) PCR using one (1) primer set for one (1) kind of pathogen

Initial denaturation (94° C., 10 minutes)—>Denaturation (94° C., 5 seconds)—>Annealing (60° C., 5 seconds)—>Extension (72° C., 5 seconds), Total 100 times

After termination of the PCR reaction, Melting curve analysis is carried out under the following conditions.

Initial Temperature: 60° C.

Final Temperature: 90° C.

Ramp Rate: ° C./sec

Exit Temperature: 40° C.

### EXAMPLE 9

### Verification of Detection Limit of Micro-PCR

9-1. Salmonella spp.

Micro-PCR composition using two kinds of primer pairs as in Example 7-1 was prepared, and micro-PCR was run under the conditions as in Example 8(1). The primers used was SEQ ID NO:1 to 4 and the specimen was Salmonella enteritidis KCCM12021.

FIG. 11 shows an analysis of the detection limit in different concentrations of Salmonella in case of nested-PCR using two kinds of primer pairs shown in SEQ ID NO:1 to 4 of Salmonella enteritidis. In FIG. 11, 1 is the result for 10⁷ CFU/ml of Salmonella enteritidis. 2 is the result for 10 CFU/ml and 3 is a negative control group.

9-2. Salmonella spp.

Micro-PCR composition using one primer pair as in Example 7-2 was prepared, and micro-PCR was run under the conditions as in Example 8(2). The primers used was SEQ ID NO:3 and 4 and the specimen was Salmonella enteritidis KCCM12021.

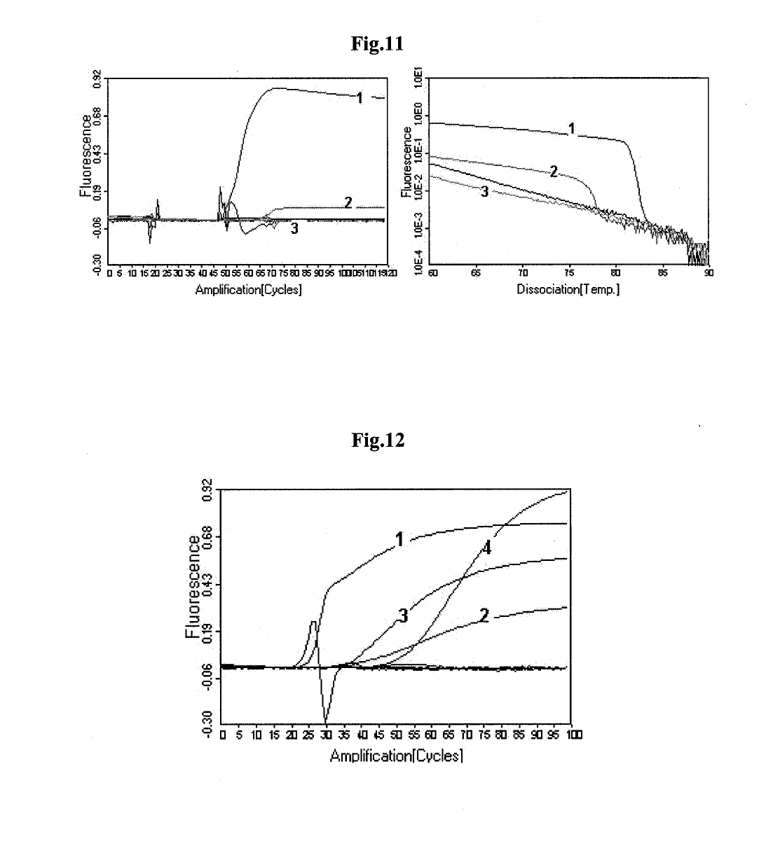

FIG. 12 shows an analysis of the detection limit in different concentrations of Salmonella in case of micro-PCR using one primer pair shown in SEQ ID NO:3 and 4 of Salmonella enteritidis. In FIG. 12, 4 is the result of 10³ CFU/ml of Salmonella enteritidis, 3 is the result of 10² CFU/ml, 2 is the result of 10¹ CFU/ml, and 1 is the result of using two kinds of primer pairs shown in SEQ ID NO:1 to 4. 2, 3 and 4 of FIG. 12 has the same start point, and it can be seen that the two kinds of the primer pairs have formed dimers with each other. Since having the same start point means there was an equal starting copy number, it can be seen that they have the same detection limit. The detection limit by the above micro-PCR is 10 CFU/ml.

9-3. Staphylococcus aureus

Micro-PCR composition using one primer pair as in Example 7-2 was prepared, and micro-PCR was run under the conditions as in Example 8(2). The primers used was SEQ ID NO:7 and 8 and specimen was Staphylococcus aureus KCCM1927.

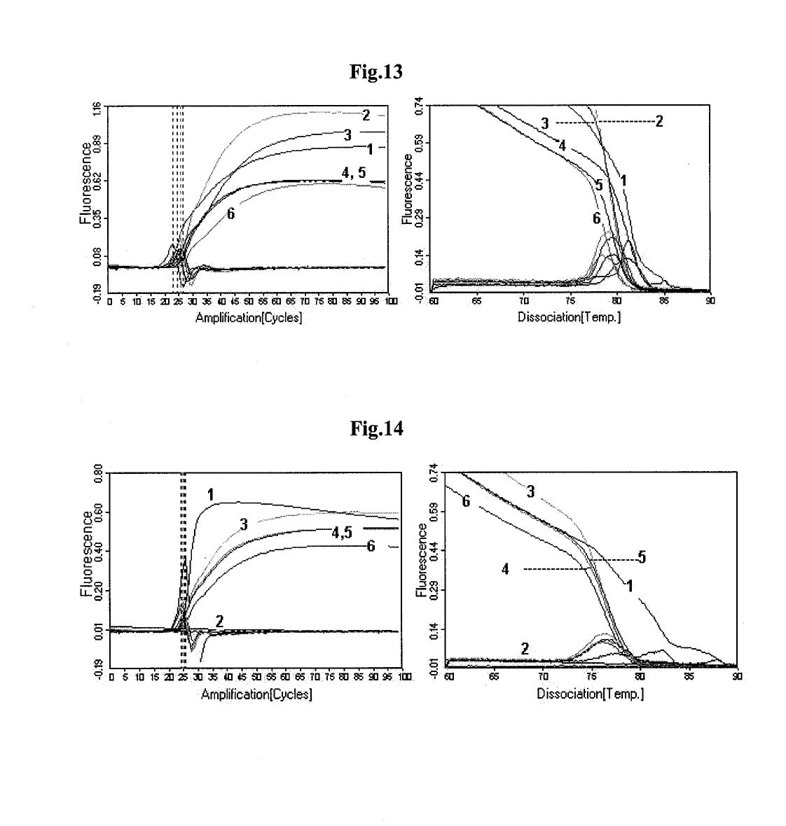

FIG. 13 shows an analysis of the detection limit in different concentrations of Salmonella in case of micro-PCR using one primer pair shown in SEQ ID NO:7 and 8 of Staphylococcus aureus. In FIG. 13, 1 is a positive control group, 2 is the result of using a primer pair shown in SEQ ID NO:5 and 6, 3 is the result of Staphylococcus aureus 10⁴ CFU/ml, 4 is the result of 10³ CFU/ml, 5 is the result of 10² CFU/ml, and 6 is the result of 10¹ CFU/ml. The detection limit of the above is 10 CFU/ml.

9-4. Escherichia coli O157

Micro-PCR composition using one primer pair as in Example 7-2 was prepared, and micro-PCR was run under the conditions as in Example 8(2). The primers used was SEQ ID NO: 11 and 12 and specimen was E. coli O157:H7 ATCC12024.

FIG. 14 shows an analysis of the detection limit in different concentrations of Salmonella in case of micro-PCR using one primer pair shown in SEQ ID NO: ______ and ______ of E. coli O157:H7. In FIG. 14, 1 is a positive control group, 2 is a negative control group, 3 is the result of E. coli O157:H7 10⁴ CFU/ml, 4 is the result of 10³ CFU/ml, and 5 is the result of 10² CFU/ml, and 6 is the result of 10¹ CFU/ml. The detection limit of the above is 10 CFU/ml.

1. A primer for detecting a pathogen comprising one or more pair of primers selected from the group consisting of:
a pair of primers for detecting Salmonella spp including a primer comprising a nucleotide sequence shown in SEQ ID NO:3, and a primer comprising a nucleotide sequence shown in SEQ ID NO:4;
a pair of primers for detecting Staphylococcus aureus including a primer comprising a nucleotide sequence shown in SEQ ID NO:7, and a primer comprising a nucleotide sequence shown in SEQ ID NO:8;
a pair of primers for detecting E. coli O157:H7 including a primer comprising a nucleotide sequence shown in SEQ ID NO:11, and a primer comprising a nucleotide sequence shown in SEQ ID NO:12;
a pair of primers for detecting E. coli O157:H7 including a primer comprising a nucleotide sequence shown in SEQ ID NO:13, and a primer comprising a nucleotide sequence shown in SEQ ID NO:14;
a pair of primers for detecting Listeria monocytogenes including a primer comprising a nucleotide sequence shown in SEQ ID NO:17, and a primer comprising a nucleotide sequence shown in SEQ ID NO:18;
a pair of primers for detecting Vibrio parahaemolyticus including a primer comprising a nucleotide sequence shown in SEQ ID NO:21, and a primer comprising a nucleotide sequence shown in SEQ ID NO:22; and
a pair of primers for detecting Vibrio parahaemolyticus including a primer comprising a nucleotide sequence shown in SEQ ID NO:23, and a primer comprising a nucleotide sequence shown in SEQ ID NO:24.

2. The primer for detecting a pathogen according to claim 1, wherein the primer further comprises a pair of primers selected from the group consisting of:
a pair of primers for detecting Salmonella spp including a primer comprising a nucleotide sequence shown in SEQ ID NO:1, and a primer comprising a nucleotide sequence shown in SEQ ID NO:2;
a pair of primers for detecting Staphylococcus aureus including a primer comprising a nucleotide sequence shown in SEQ ID NO:5, and a primer comprising a nucleotide sequence shown in SEQ ID NO:6;
a pair of primers for detecting E. coli O157:H7 including a primer comprising a nucleotide sequence shown in SEQ ID NO:9, and a primer comprising a nucleotide sequence shown in SEQ ID NO:10;
a pair of primers for detecting Listeria monocytogenes including a primer comprising a nucleotide sequence shown in SEQ ID NO:15, and a primer comprising a nucleotide sequence shown in SEQ ID NO:16; and
a pair of primers for detecting Vibrio parahaemolyticus including a primer comprising a nucleotide sequence shown in SEQ ID NO:19, and a primer comprising a nucleotide sequence shown in SEQ ID NO:20.

3. A method of detecting a pathogen by using PCR with primer(s) according to claim 1.

4. The method of detecting a pathogen according to claim 3, wherein the PCR is a thermal block PCR or a micro-PCR.

5. The method of detecting a pathogen according to claim 3, wherein a second round of PCR is performed by using the primer according to claim 2, following the PCR by using the primer in claim 1.

6. The method of detecting a pathogen according to claim 3, wherein the PCR is performed by simultaneously using the primers according to claim 1 and claim 2.

7. The method of detecting a pathogen according to claim 3, wherein the detection method is performed by simultaneously detecting at least two pathogens through multiplex PCR by using at least two primer pairs for detecting different pathogens.

8. A kit for detecting a pathogen comprising a PCR primer for detecting a pathogen through PCR, a reaction buffer, and Taq DNA polymerase, and the PCR primer is a primer set(s) according to claim 1.

9. A method of detecting a pathogen by using PCR with primer(s) according to claim 2.

10. The method of detecting a pathogen according to claim 9, wherein the PCR is a thermal block PCR or a micro-PCR.

11. The method of detecting a pathogen according to claim 9, wherein a second round of PCR is performed by using the primer according to claim 2, following the PCR by using the primer in claim 1.

12. The method of detecting a pathogen according to claim 9, wherein the PCR is performed by simultaneously using the primers according to claim 1 and claim 2.

13. The method of detecting a pathogen according to claim 9, wherein the detection method is performed by simultaneously detecting at least two pathogens through multiplex PCR by using at least two primer pairs for detecting different pathogens.

14. A kit for detecting a pathogen comprising a PCR primer for detecting a pathogen through PCR, a reaction buffer, and Taq DNA polymerase, and the PCR primer is a primer set(s) according to claim 2.


SEQUENCE LISTING (24 sequences)

SEQ ID NO: 1 - DNA, 24 bp, Artificial Sequence
  P1 primer
gaatcctcagtttttcaacgtttc

SEQ ID NO: 2 - DNA, 24 bp, Artificial Sequence
  P2 primer
tagccgtaacaaccaatacaaatg

SEQ ID NO: 3 - DNA, 20 bp, Artificial Sequence
  P′1 primer
tcgtcattccattacctacc

SEQ ID NO: 4 - DNA, 20 bp, Artificial Sequence
  P′2 primer
atcggcttcaatcaagataa

SEQ ID NO: 5 - DNA, 24 bp, Artificial Sequence
  P3 primer
aatttaacagctaaagagtttggt

SEQ ID NO: 6 - DNA, 24 bp, Artificial Sequence
  P4 primer
ttcattaaagaaaaagtgtacgag

SEQ ID NO: 7 - DNA, 20 bp, Artificial Sequence
  P′3 primer
aatgctttccgtcattttgc

SEQ ID NO: 8 - DNA, 20 bp, Artificial Sequence
  P′4 primer
agcttttgctgatcgtgatg

SEQ ID NO: 9 - DNA, 24 bp, Artificial Sequence
  P5 primer
gacagcagttataccactctgcaa

SEQ ID NO: 10 - DNA, 24 bp, Artificial Sequence
  P6 primer
gacgaaattctctctgtatctgcc

SEQ ID NO: 11 - DNA, 20 bp, Artificial Sequence
  P′5 primer
ggtgttccttttggctgaag

SEQ ID NO: 12 - DNA, 20 bp, Artificial Sequence
  P′6 primer
tgacgactgatttgcattcc

SEQ ID NO: 13 - DNA, 20 bp, Artificial Sequence
  P′′5 primer
ttctgagcaatcggtcactg

SEQ ID NO: 14 - DNA, 20 bp, Artificial Sequence
  P′′6 primer
tatatcagtgcccggtgtga

SEQ ID NO: 15 - DNA, 24 bp, Artificial Sequence
  P7 primer
ctggcacaaaattacttacaacga

SEQ ID NO: 16 - DNA, 24 bp, Artificial Sequence
  P8 primer
aactactggagctgcttgtttttc

SEQ ID NO: 17 - DNA, 20 bp, Artificial Sequence
  P′7 primer
ttggtgcaactggagtgctt

SEQ ID NO: 18 - DNA, 20 bp, Artificial Sequence
  P′8 primer
agcaggtgcagcttgttgag

SEQ ID NO: 19 - DNA, 24 bp, Artificial Sequence
  P9 primer
ctcatttgtactgttgaacgccta

SEQ ID NO: 20 - DNA, 24 bp, Artificial Sequence
  P10 primer
aatagaaggcaaccagttgttgat

SEQ ID NO: 21 - DNA, 20 bp, Artificial Sequence
  P′9 primer
cttctgacgcaatcgttgaa

SEQ ID NO: 22 - DNA, 20 bp, Artificial Sequence
  P′10 primer
gacctgcgaaaatacgcaat

SEQ ID NO: 23 - DNA, 20 bp, Artificial Sequence
  P′′9 primer
tgatttgcgggtgatttaca

SEQ ID NO: 24 - DNA, 20 bp, Artificial Sequence
  P′′10 primer
gagattccgctgggtttgta